(19) United States
(12) Patent Application Publication
(10) Pub. No.: US 2026/0345383 A1
(43) Pub. Date: Nov. 5, 2026

(54) SURGICAL MONITORING

(71) Applicant: ALCON INC., Fribourg (CH)

(72) Inventors: Zhuoran WU, Fort Worth, TX (US); Lu YIN, Fort Worth, TX (US); Sinchan BHATTACHARYA, Fort Worth, TX (US); Aakash NAGARAJAN, Fort Worth, TX (US); Ramesh SARANGAPANI, Fort Worth, TX (US); Nikhil SCARIA, Fort Worth, TX (US); Vignesh SURESH, Fort Worth, TX (US)

(21) Appl. No.: 19/650,256

(22) Filed: Apr. 16, 2026

Related U.S. Application Data
(60) Provisional application No. 63/794,906, filed on Apr. 25, 2025.

(51) Int. Cl. A61F 9/007; A61B 3/00; A61B 3/14

(52) U.S. Cl. CPC A61F 9/007; A61B 3/0025; A61B 3/14

(57) ABSTRACT

A method of monitoring during a medical procedure may include capturing, by a first image sensor, a first image of an eye during a surgery on the eye and capturing, by a second image sensor, a second image of the eye during the surgery on the eye. In some embodiments, the second image may be captured at the same time as the first image. The method may also include analyzing, by a processing system during the surgery, the first image and the second image to determine one or more three-dimensional features of the eye and predicting, by the processing system during the surgery, one or more conditions of the eye based on the one or more three-dimensional features of the eye. The method may further include presenting an indication of the one or more conditions during the surgery.

### CROSS-REFERENCE TO RELATED APPLICATION

This application claims priority to U.S. Provisional Application No. 63/794,906, filed on Apr. 25, 2025, which is incorporated herein in its entirety.

### FIELD

The embodiments discussed herein relate to a monitoring during a surgery.

### BACKGROUND

Cataract surgery is a procedure in which the crystalline lens of the eye is removed by phacoemulsification and replaced with an artificial intraocular lens implanted within the posterior lens capsule. During the procedure, the surgeon operates under a surgical microscope that provides a magnified view of the anterior segment of the eye. Intraoperative monitoring of ocular structures, including the pupil, iris, and anterior lens capsule, relies on the direct visual assessment of the surgeon.

The subject matter claimed herein is not limited to embodiments that solve any disadvantages or that operate only in environments such as those described above. Rather, this background is only provided to illustrate one example technology area where some embodiments described herein may be practiced.

### SUMMARY

According to an aspect of at least one embodiment, a method of monitoring during a medical procedure may include capturing, by a first image sensor, a first image of an eye during a surgery on the eye and capturing, by a second image sensor, a second image of the eye during the surgery on the eye. In some embodiments, the second image may be captured at the same time as the first image. The method may also include analyzing, by a processing system during the surgery, the first image and the second image to determine one or more three-dimensional features of the eye and predicting, by the processing system during the surgery, one or more conditions of the eye based on the one or more three-dimensional features of the eye. The method may further include presenting an indication of the one or more conditions during the surgery.

The object and advantages of the embodiments will be realized and achieved at least by the elements, features, and combinations particularly pointed out in the claims. It is to be understood that both the foregoing general description and the following detailed description are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1a illustrates an example environment for surgical monitoring;

FIG. 1b illustrates an example presentation on a display;

FIG. 2 illustrates an example process flow for surgical monitoring;

FIG. 3 illustrates a flowchart of an example method of surgical monitoring;

FIG. 4 illustrates a flowchart of another example method of surgical monitoring;

FIG. 5 is a block diagram of an example computing system for generative artificial intelligence (AI); and

FIG. 6 is a block diagram of an example computing device, all in accordance with at least some embodiments of the present disclosure.

### DETAILED DESCRIPTION

During ophthalmic surgical procedures, a surgeon may operate on structures within the eye while monitoring the surgical field through a microscope or a heads-up display system. Under existing approaches, a surgeon may rely primarily on direct visual observation to detect changes in the eye that may indicate a condition or complication that, if left undetected, may result in adverse patient outcomes.

For example, a pupil of an eye may change in size, shape, position, and movement during a surgery. Some of the changes to the pupil may be normal. Alternatively, some changes that occur to the pupil may be indicative of a complication that is occurring during the surgical procedure or an underlying condition of the eye that may direct that different protocols occur during the surgical procedure to increase the probability of a better outcome. For example, during cataract surgery, the pupil may begin to constrict unexpectedly, shift asymmetrically toward one side, or exhibit irregular oscillatory behavior. Each of these changes may signal a distinct clinical condition, such as intraoperative floppy iris syndrome, zonular weakness, iris prolapse, or anterior chamber instability.

Some monitoring systems may attempt to assist a surgeon in detecting changes in the eye. However, the monitoring systems may be limited to two-dimensional image-based monitoring of the eye. In some circumstances, two-dimensional image-based monitoring of the eye may fail to distinguish between apparent shape changes caused by the tilt of the eye relative to the imaging axis and true anatomical changes in the eye. For example, a pupil that appears elliptical in a two-dimensional image may, in three-dimensional space, remain circular but tilted, or may be elongated due to zonular fiber disruption. Without three-dimensional analysis, a monitoring system may be unable to distinguish between a zonular fiber disruption and a tilted pupil.

Further limitations of existing approaches relate to the scope of parameters monitored. Prior monitoring systems may track pupil size as a primary or sole metric. However, pupil size alone may be insufficient to characterize the full range of clinically meaningful events. For example, the position of the pupil centroid relative to the limbus, the rate at which the pupil diameter changes over time, the shape of the pupil as characterized by ellipticity and axis ratio, the stability of the pupil center in three-dimensional space, and the behavior of the capsulorhexis edge relative to the pupil center each provide distinct and complementary information about the state of the eye during surgery. No prior system may integrate multiple three-dimensional features of the eye into a framework that predicts conditions of the eye during a surgical procedure.

The present disclosure relates generally to systems and methods for real-time, intraoperative monitoring of the eye during ophthalmic surgery. In these and other embodiments, a medical system may capture synchronized images of the eye from two separate image sensors during surgery. A processing system may analyze the images to determine three-dimensional features of the eye, including three-dimensional features of the pupil. In some embodiments, the processing system may also determine three-dimensional dynamic features of the eye. The three-dimensional dynamic features of the eye may allow the processing system to characterize the current state of the eye as well as a rate and direction of change of the state throughout the surgical procedure.

Using the three-dimensional features and/or the three-dimensional dynamic features, the processing system may predict one or more conditions of the eye and may present an indication of those conditions to the surgeon during the surgical procedure. By analyzing the eye in three dimensions rather than two, the processing system may distinguish between changes attributable to normal eye movement and changes attributable to conditions that may be addressed during the surgery.

As a result, the systems and methods of the present disclosure may provide real-time, quantitative information about the state of the eye that may otherwise be undetectable through direct visual observation alone. Based on the additional information, the surgeon may adjust surgical technique, modify surgical device parameters, and/or deploy additional devices in response to the additional information to help reduce the probability of undesirable intraoperative and postoperative outcomes.

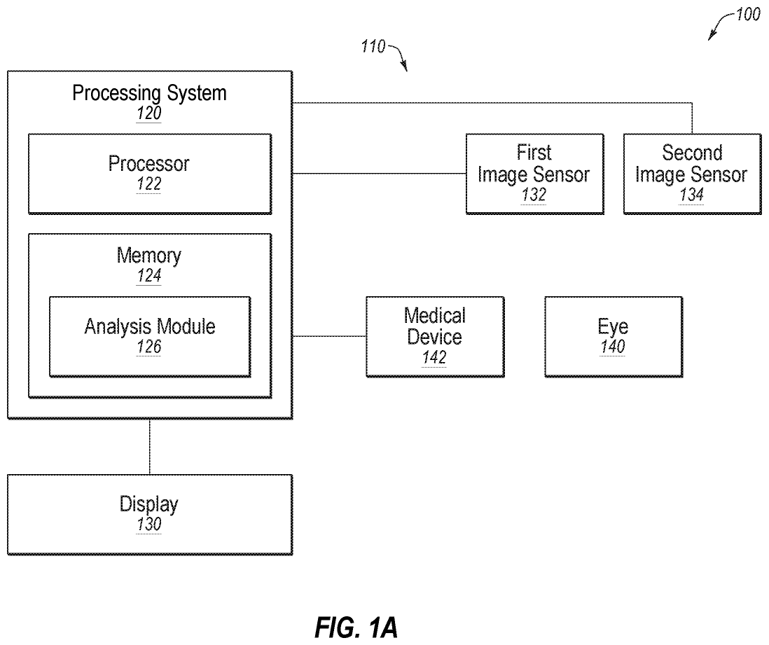

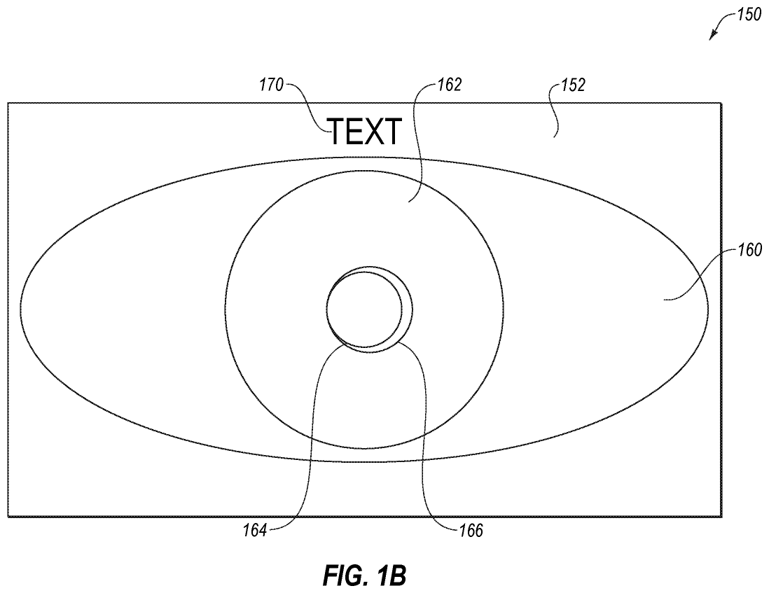

FIG. 1A illustrates an example environment 100 for real-time, intraoperative monitoring of an eye during ophthalmic surgery. The environment 100 may be arranged in accordance with at least one embodiment described in the present disclosure. The environment 100 may include a medical system 110 and an eye 140. The medical system 110 may include a processing system 120, a display 130, a first image sensor 132, a second image sensor 134, and a medical device 142. The processing system 120 may include a processor 122, a memory 124, and an analysis module 126.

In some embodiments, the medical system 110 may operate during a medical procedure. For example, the medical system 110 may operate during an ophthalmic surgical procedure, such as cataract surgery, vitreoretinal surgery, glaucoma surgery, or laser eye surgery. During such a procedure, the first image sensor 132 and the second image sensor 134 may each capture images of the eye 140. The processing system 120 may analyze the captured images to determine three-dimensional features of the eye 140 and may predict one or more conditions of the eye 140 based on those features. The display 130 may present an indication of the predicted conditions to a surgeon during the surgical procedure.

The first image sensor 132 may be configured to capture first images/video of the eye 140 during surgery on the eye 140. In some embodiments, the first image sensor 132 may be included in a camera mounted to a stereo surgical microscope, such as a camera associated with a left optical channel of the stereo surgical microscope. In these and other embodiments, the first image sensor 132 may capture video of the eye 140 at a frame rate sufficient for real-time analysis, for example at a rate of thirty to one hundred twenty frames per second. In these and other embodiments, each frame of the video may be an image, and the terms frame and/or image may be used interchangeably in the current disclosure. As such, the first image sensor 132 may be described as capturing an image or video. In these and other embodiments, an anterior segment or other portions of the eye 140 may be captured in the video generated by the first image sensor 132.

In some embodiments, the first image sensor 132 may be pre-calibrated with known intrinsic parameters, including an intrinsic matrix K, a focal length f, a principal point having coordinates Cx and Cy, and radial and tangential lens distortion coefficients k1, k2, p1, and p2. In some embodiments, the first image sensor 132 may transmit each captured image to the processing system 120 for downstream analysis.

In some embodiments, the second image sensor 134 may be configured to capture a second images/video of the eye 140 during surgery on the eye 140. In some embodiments, the second image sensor 134 may be included in a camera associated with a right optical channel of the stereo surgical microscope. The second image sensor 134 may share the same intrinsic calibration parameters as the first image sensor 132 or may have separately characterized parameters. In some embodiments, the second image sensor 134 may also transmit each captured image to the processing system 120.

In some embodiments, the second image sensor 134 may be spatially separated from the first image sensor 132 by a stereo baseline distance B. In these and other embodiments, the spatial separation between the first image sensor 132 and the second image sensor 134 may enable the processing system 120 to compute stereo disparity between images from which three-dimensional coordinates of anatomical structures within the eye 140 may be derived.

In some embodiments, the second images or frames of the second video may be captured at the same time as the first image or frames of the first video captured by the first image sensor 132. A first image/frame captured at the same time as the second image may be referred to as an image pair. In some embodiments, capturing the second image at the same time as the first image may indicate that the first image sensor 132 and the second image sensor 134 capture the image pair within the same video frame interval, such that the images in the image pair represent the eye 140 at a common timeframe during the surgical procedure. A length of the common time frame may be between approximately two milliseconds and sixty milliseconds. Alternately or additionally, the length of the common time frame may correspond to a frame rate of approximately thirty to one hundred twenty frames per second. In these and other embodiments, the second image being captured at the same time as the first image may create a temporal correspondence between the first image and the second image which may enable the processing system 120 to compute stereo disparity from a spatially separated but temporally synchronized images.

The synchronized capture of images by the first image sensor 132 and the second image sensor 134 and the spatial separation between the first image sensor 132 and the second image sensor 134 may allow the processing system 120 to reconstruct the three-dimensional geometry of the eye 140 during the surgical procedure. For example, the first image sensor 132 and the second image sensor 134 may each capture a respective image of the pupil of the eye 140 during a common timeframe. The processing system 120 may then compare the positions of the pupil centroid in the first image and the second image to compute a disparity value d, from which a depth value Z may be derived using the relationship Z=(f·B)/d. From the depth value Z and the intrinsic parameters of the first image sensor 132, the three-dimensional coordinates X and Y of the pupil center may be computed, yielding a three-dimensional pupil center position P=(X,Y,Z) in the physical space of the surgical field.

In some embodiments, the processing system 120 may be configured to analyze the first video and the second video to determine one or more three-dimensional features of the eye 140. The processing system 120 may include the processor 122, the memory 124, and the analysis module 126. The processor 122 may execute instructions stored in the memory 124 to perform image analysis, three-dimensional reconstruction, feature extraction, and condition prediction. The memory 124 may store the analysis module 126, which may include software instructions and/or trained model parameters used to perform the operations described herein. In some embodiments, the processor 122 may include a graphics processing unit (GPU) or other parallel processing hardware capable of executing deep learning inference at real-time frame rates.

The analysis module 126 may be configured to perform several operations on the images received from the first image sensor 132 and the second image sensor 134. In some embodiments, the analysis module 126 may apply a trained segmentation model to each image pair to produce segmentation masks for anatomical structures of the eye 140. The anatomical structures that may be segmented include the pupil, the limbus, the sclera, and the capsulorhexis (rhexis) edge, among other structures of the eye. The segmentation model may be a convolutional neural network having an encoder-decoder architecture, trained on annotated intraoperative ophthalmic surgical image datasets. For each frame, the segmentation model may output a pupil mask, a limbus mask, a sclera mask, and a rhexis mask, among other masks. Each mask produced by the segmentation model may represent a spatial map in which pixels, assigned a value of one, correspond to a particular anatomical structure and pixels assigned a value of zero correspond to all other regions of the image. As such, the masks may each delineate a distinct anatomical structure of the eye 140 within an image.

In some embodiments, the analysis module 126 may further apply a pixel-to-physical unit scale factor to convert all pixel-space measurements into physical units of millimeters (mm). In these and other embodiments, the scale factor may be computed from the limbus mask by detecting the limbus boundary using a contour detection algorithm and measuring the horizontal pixel extent of the limbus, denoted Wlimbus, pixel, as the difference between the maximum and minimum horizontal pixel coordinates of the limbus boundary. The scale factor may then be computed as s=Wlimbus,real/Wlimbus,pixel, where Wlimbus,real represents the known true anatomical limbus width in millimeters. The scale factor may be distributed to all parameter computation operations within the analysis module 126 to enable conversion of pixel measurements into physical dimensions.

Using the segmentation masks and the scale factor, the analysis module 126 may extract two-dimensional and three-dimensional features of the eye 140. In some embodiments, the two-dimensional features extracted from a single image pair may include features of the pupil, such as various dimensions of the pupil.

In some embodiments, the analysis module 126 may identify and determine the two-dimensional features of the eye 140 by applying computational methods to the segmentation masks and contour coordinates produced by the segmentation model. The analysis module 126 may process the binary pixel values of each segmentation mask to locate boundary coordinates of each anatomical structure, fit geometric primitives such as ellipses to those boundary coordinates, and compute scalar and vector quantities from the resulting geometric parameters.

In some embodiments, the different dimensions of the pupil that may be determined may include a pupil centroid position, a pupil major axis length, a pupil minor axis length, a pupil orientation angle, a pupil width, a pupil height, a measured pupil diameter, a physical pupil diameter corrected for lens distortion, a pupil-to-limbus ratio, a pupil circumference, a pupil axis ratio, a pupil confidence score, a pupil outline confidence score, a rhexis edge centroid position, a rhexis-to-pupil center distance, and a rhexis edge orientation angle, among other features of structural elements of the eye. Each of those features may characterize the geometric and spatial state of the eye 140 at a single point in time during the surgical procedure.

In some embodiments, the analysis module 126 may also extract three-dimensional features from each image pair produced from the first image and the second image. In some embodiments, the analysis module 126 may identify and determine the three-dimensional features of the eye 140 by applying stereo triangulation and geometric reconstruction methods to the segmentation masks and contour coordinates produced by the segmentation model for each image pair. For example, the analysis module 126 may compute disparity values, e.g. the difference in positions, between corresponding anatomical feature locations in the first image and the second image. The analysis module 126 may then derive depth values from the disparity values using the stereo calibration parameters of the first image sensor 132 and the second image sensor 134. Depth values may be derived from the disparity values by applying the relationship Z=(f·B)/d, where Z represents the depth value, f represents the focal length of the first image sensor 132, B represents the stereo baseline distance between the first image sensor 132 and the second image sensor 134, and d represents the disparity value computed from the corresponding anatomical feature locations in the first image and the second image. From the depth value Z and the intrinsic parameters of the first image sensor 132 and the second image sensor 134, the three-dimensional coordinates X and Y of each anatomical feature location may be derived using the relationships X=(x−Cx)·Z/f and Y=(y−Cy)·Z/f, where (Cx, Cy) represents the principal point of the first image sensor 132.

After deriving the depth values, the analysis module 126 may project the resulting depth values together with the two-dimensional pixel coordinates into three-dimensional Cartesian coordinates in the physical space of the surgical field. From the three-dimensional coordinates of anatomical boundary points, the analysis module 126 may fit geometric primitives such as planes and ellipses to the reconstructed three-dimensional point sets and may compute scalar and vector quantities from the resulting three-dimensional geometric parameters, including tilt angles and physical axis lengths in three-dimensional space. For example, the three-dimensional features extractable from a single image pair may include a three-dimensional pupil center position, a three-dimensional pupil major axis length, a three-dimensional pupil minor axis length, a pupil tilt angle relative to the imaging plane, a three-dimensional pupil axis ratio, a three-dimensional pupil circumference, a three-dimensional segmentation volume of the pupil point cloud, a capsular bag elevation derived from the three-dimensional position of the rhexis edge, and a three-dimensional rhexis edge centroid position.

In some embodiments, the analysis module 126 may also compute three-dimensional dynamic features of the eye 140 from multiple image pairs captured over time. The three-dimensional dynamic features may characterize the temporal behavior of anatomical structures of the eye 140 throughout the surgical procedure. Temporal behavior may refer to the manner in which three-dimensional features of the eye 140 change over successive image pairs captured throughout the surgical procedure. The processing system 120 may analyze temporal behavior to detect rates of change, directional trends, and/or oscillatory patterns in anatomical structures of the eye 140 that may indicate developing intraoperative conditions before those conditions become visually apparent to the surgeon.

To determine the three-dimensional dynamic features, the analysis module 126 may track the three-dimensional position of the three-dimensional features across successive image pairs and may compute displacement vectors between consecutive positions. From the sequence of displacement vectors, the analysis module 126 may derive cumulative path length, average velocity, and/or instantaneous acceleration of the three-dimensional features in three-dimensional space. In some embodiments, the analysis module 126 may also compute a measure of positional stability by evaluating the statistical spread of the three-dimensional features positions over a defined temporal window during the surgical procedure.

As an example, the analysis module 126 may track the three-dimensional position of a center of a pupil and determine a cumulative path length, average velocity, and/or instantaneous acceleration of the center of a pupil in three-dimensional space over time. As another example, in some embodiments, the analysis module 126 may apply analogous temporal tracking to the three-dimensional positions of the rhexis edge centroid, yielding displacement, elevation change, and orientation rotation of the rhexis edge over time.

In some embodiments, the analysis module 126 may also compute averaged or cumulative representations of select features across multiple image pairs. For example, the analysis module 126 may compute a mean displacement of the rhexis edge centroid from a baseline position established during an early stable phase of the surgical procedure, a mean upward shift of the rhexis edge in the depth dimension, and a mean change in rhexis edge orientation angle relative to a baseline orientation. As another example, the analysis module 126 may further compute the standard deviation of the distance between the rhexis edge centroid and the pupil centroid over a series of image pairs, yielding a measure of relative positional variability between the two anatomical structures. Each of the three-dimensional dynamic features derived from multiple image pairs may represent a distinct aspect of the temporal state of the eye 140 and may provide information about the rate, direction, and consistency of change in the anatomical structures of the eye 140 throughout the surgical procedure.

The processing system 120 may be configured to predict one or more conditions of the eye 140 based on the two-dimensional features, three-dimensional features, and/or the three-dimensional dynamic features of the eye 140. In some embodiments, the conditions predicted by the processing system 120 may include risk scores that quantify the likelihood of a particular intraoperative or postoperative event, as well as specific named conditions such as intraoperative floppy iris syndrome, zonular weakness, iris prolapse, or anterior chamber instability. Generally, the term “conditions” as used in the present disclosure may refer to any of the risk scores, predicted probabilities of specific events, or identified clinical states of the eye 140 during the surgical procedure.

In some embodiments, the processing system 120 may predict one or more conditions of the eye 140 by evaluating one or more prediction conditions against the two-dimensional features, three-dimensional features, and/or three-dimensional dynamic features extracted by the analysis module 126. A prediction condition may define a relationship between one or more extracted features and a threshold value, a baseline value, a rate of change, a directional trend, or a combination thereof, such that satisfaction of the prediction condition by the extracted features may indicate a likelihood that a particular condition of the eye 140 exists or may develop during the surgical procedure. As used herein, thresholds may be, for example, fixed values, adaptive values, patient-normalized values, or values output by a trained model.

For example, a prediction condition may be defined with respect to a single feature, such as a deviation of a three-dimensional pupil diameter from a baseline diameter exceeding a predetermined magnitude. As another example, a prediction condition may be defined with respect to multiple features evaluated in combination, such that no single feature alone may satisfy the prediction condition but a weighted aggregation of multiple features may satisfy the prediction condition when the aggregated value exceeds a predetermined threshold. In some embodiments, the prediction conditions may be established prior to the surgical procedure based on population-level clinical data, and may be further adjusted based on patient-specific data, including patient age, medication history, ocular pigmentation, prior ocular trauma, and prior surgical history, such that the prediction conditions applied during the surgical procedure may reflect both population-level and patient-individualized parameters. In some embodiments, satisfaction of one or more prediction conditions may cause the processing system 120 to generate an indication of the predicted condition for presentation on the display 130 during the surgical procedure.

Alternately or additionally, in some embodiments, the processing system 120 may predict one or more conditions of the eye 140 by applying a predictive model to the two-dimensional features, three-dimensional features, and/or three-dimensional dynamic features extracted by the analysis module 126.

In some embodiments, the processing system 120 may apply the predictive model to a feature vector assembled from the two-dimensional features, three-dimensional features, and/or three-dimensional dynamic features of the eye 140. The feature vector may represent the state of the eye 140 at a single point in time or may represent the temporal behavior of the eye 140 across multiple image pairs captured throughout the surgical procedure. The analysis module 126 may assemble the feature vector by collecting the scalar and vector quantities derived from the segmentation masks and stereo reconstruction operations described above and may pass the assembled feature vector to the predictive model for evaluation.

In some embodiments, the predictive model applied by the processing system 120 may be a weighted regression model that assigns a weight to each feature in the feature vector. The processing system 120 may compute a composite risk score by combining the weighted features into a scalar value. The weights applied to each feature may be derived from training data comprising historical intraoperative parameter datasets paired with known postoperative clinical outcomes. In some embodiments, the weights may be determined separately for each condition being predicted, such that a distinct set of weights may be applied when predicting a first condition and a different set of weights may be applied when predicting a second condition.

In some embodiments, the processing system 120 may compare the composite risk score produced by the predictive model against a predetermined threshold. When the composite risk score exceeds the predetermined threshold, the processing system 120 may generate an indication of the predicted condition for presentation to the surgeon.

In some embodiments, the predictive model may incorporate two-dimensional features of the eye 140 to predict conditions. The two-dimensional features that may contribute to the prediction may include the measured pupil diameter, the rate of change of the pupil diameter over time, the pupil axis ratio, the pupil-to-limbus ratio, the pupil outline confidence score, and the position of the pupil centroid relative to the limbus, among other two-dimensional features. In these and other embodiments, the processing system 120 may evaluate deviations of each two-dimensional feature from a baseline value established during an early stable phase of the surgical procedure and may weight those deviations according to the trained weights of the predictive model to produce a contribution to the composite risk score.

In some embodiments, the predictive model may also incorporate three-dimensional features of the eye 140 to predict conditions. The three-dimensional features that may contribute to the prediction may include the three-dimensional pupil major axis length, the three-dimensional pupil minor axis length, the pupil tilt angle relative to the imaging plane, the three-dimensional pupil axis ratio, and the capsular bag elevation derived from the three-dimensional position of the rhexis edge, among other three-dimensional features. In these and other embodiments, the processing system 120 may evaluate each three-dimensional feature relative to a baseline and may weight the resulting deviations to produce a contribution to the composite risk score.

In some embodiments, the predictive model may further incorporate three-dimensional dynamic features of the eye 140 to predict conditions. The three-dimensional dynamic features that may contribute to the prediction may include the cumulative path length of the three-dimensional pupil center position over time, the average velocity of the three-dimensional pupil center, the instantaneous acceleration of the three-dimensional pupil center, the standard deviation of the three-dimensional pupil center position over a defined temporal window, the mean displacement of the rhexis edge centroid from a baseline position, the mean elevation change of the rhexis edge in the depth dimension, the mean change in rhexis edge orientation angle, and the standard deviation of the distance between the rhexis edge centroid and the pupil centroid, among other three-dimensional dynamic features. The processing system 120 may weight each three-dimensional dynamic feature according to the trained weights of the predictive model and may combine the weighted values to produce a contribution to the composite risk score.

In some embodiments, the processing system 120 may apply a separate predictive model for each condition being predicted. For example, a first predictive model may be applied to predict a risk score associated with intraoperative floppy iris syndrome, and a second predictive model may be applied to predict a risk score associated with zonular weakness. Each predictive model may assign different weights to the two-dimensional features, three-dimensional features, and three-dimensional dynamic features of the eye 140, reflecting the distinct clinical signatures associated with each condition. The processing system 120 may evaluate each predictive model independently and may generate a separate risk score for each condition.

In some embodiments, the processing system 120 may apply a survival model, such as a Cox proportional hazards model, to correlate the two-dimensional features, three-dimensional features, and/or three-dimensional dynamic features of the eye 140 with postoperative clinical outcomes. The survival model may receive the feature vector as covariates and may output a hazard function representing the predicted risk of a postoperative complication at a future time point. In some embodiments, the processing system 120 may alternatively apply a linear regression model to predict a continuous postoperative outcome measure as a function of the intraoperative features of the eye 140.

In some embodiments, the processing system 120 may establish a patient-specific baseline by monitoring the two-dimensional features, three-dimensional features, and/or three-dimensional dynamic features of the eye 140 during an early stable phase of the surgical procedure before significant manipulation of the eye 140 has occurred. The processing system 120 may compute deviations of each feature from the patient-specific baseline when evaluating the predictive model, rather than relying solely on population-level reference values. In some embodiments, the patient-specific baseline may allow the processing system 120 to account for individual variation in pupil reactivity and anatomy, thereby reducing the rate of false positive and false negative predictions for a given patient.

In some embodiments, the processing system 120 may predict one or more conditions of the eye 140 by applying a neural network or other artificial intelligence model to the feature vector assembled from the two-dimensional features, three-dimensional features, and/or three-dimensional dynamic features of the eye 140. For example, the neural network may be a recurrent neural network having a long short-term memory architecture, configured to receive a time-series sequence of feature vectors as input and to output a predicted probability for each condition being monitored during the surgical procedure. In some embodiments, the neural network may be a gradient boosted tree model or a transformer-based architecture trained on annotated intraoperative surgical datasets paired with known postoperative clinical outcomes. The neural network may receive the feature vector as input and may output a predicted probability for each condition. In some embodiments, the neural network may be trained on historical intraoperative parameter datasets paired with known postoperative clinical outcomes, and the trained weights of the neural network may encode the relationships between the two-dimensional features, three-dimensional features, and three-dimensional dynamic features of the eye 140 and the likelihood of each condition.

In some embodiments, the prediction of one or more conditions of the eye 140 may be further based on patient data and/or surgical data. In these and other embodiments, patient data may include information about a patient undergoing the surgery. For example, the information may include a patient age and sex, data regarding the eye of the patient include an axial length, ocular pigmentation grade, and a keratometry measurements. Alternately or additionally, the information may include medical history of the patient including prior ocular trauma history and prior ocular surgery history, among other history information.

Surgical data may include a parameter of the medical device 142 being used during the surgery. For example, the medical device 142 may be a phacoemulsification machine, an iris expansion device, an intraocular lens implantation system, a vitreoretinal surgical instrument, and glaucoma surgical devices, among other devices used during ophthalmic surgical procedures. In these and other embodiments, the medical device 142 may provide one or more parameters to the processing system 120 for use in predicting one or more conditions of the eye 140. For example, the medical device 142 may output real-time operational parameters including intraocular pressure (IOP), vacuum level, aspiration flow rate, ultrasound power delivery, and surge event flags. The processing system 120 may receive these parameters from the medical device 142 and incorporate them into the prediction of conditions of the eye 140. For example, an elevated IOP setting from the medical device 142 may cause the pupil to dilate, and the processing system 120 may account for this machine-induced dilation when evaluating whether observed pupil size changes reflect a negative condition or a normal response to machine parameters.

In some embodiments, in response to the prediction of one or more conditions of the eye 140, a parameter of the medical device 142 may be adjusted. For example, if the processing system 120 predicts anterior chamber instability based on rapid oscillatory changes in the three-dimensional pupil center position combined with elevated aspiration parameters from the medical device 142, the aspiration flow rate or IOP setting of the medical device 142 may be reduced to address the predicted condition.

The display 130 may be configured to present one or more of the first image and the second image of the eye 140 during the surgical procedure. For example, the display 130 may be a surgical monitor or a heads-up display (HUD) integrated with the stereo surgical microscope. In these and other embodiments, the processing system 120 may present the images captured by the first image sensor 132 and the second image sensor 134 during the surgery. In these and other embodiments, the processing system 120 may also direct an indication of the one or more predicted conditions to the display 130 for presentation concurrently with images captured by the first image sensor 132 and the second image sensor 134 during the surgery.

In some embodiments, the indication may take the form of an overlay rendered on the live surgical video feed. The overlay may include colored contour lines delineating the segmented masks, readouts of values for two-dimensional features, three-dimensional features, and/or three-dimensional dynamic features. The overlay may further include alert banners regarding a condition when a condition is predicted. In some embodiments, the display 130 may also present a comparison overlay showing the current pupil boundary contour superimposed on the pupil boundary contour from a prior time point, with regions of pupil expansion rendered in a first color and regions of pupil contraction rendered in a second color, enabling the surgeon to perceive the direction and magnitude of pupil change at a glance. In some embodiments, the processing system 120 may also generate auditory alerts through an audio output device when a condition is predicted.

Examples of conditions that may be predicted by the medical system 110 now follow. For example, in some embodiments, the processing system 120 may predict a condition of the eye 140 corresponding to iris prolapse. Iris prolapse may occur when iris tissue begins to herniate through a corneal incision during the surgical procedure. The processing system 120 may detect early indicators of iris prolapse by evaluating a combination of two-dimensional features, three-dimensional features, and three-dimensional dynamic features of the eye 140. For example, the analysis module 126 may monitor the pupil centroid displacement vector relative to the limbus centroid, the pupil axis ratio, and/or the rate of change of the pupil shape over successive image pairs. When the processing system 120 detects a progressive deviation of the pupil shape from circularity, combined with an asymmetric shift of the pupil centroid toward a wound-adjacent region and an elevated aspiration parameter from the medical device 142, the processing system 120 may generate an indication of elevated iris prolapse risk for presentation on the display 130 during the surgical procedure.

In another example, in some embodiments, the processing system 120 may predict a condition of the eye 140 corresponding to underlying zonular instability. Zonular instability may arise when zonular fibers supporting the capsular bag become weakened or disrupted during the surgical procedure, causing the capsular bag, iris, and pupil to shift asymmetrically toward the side of fiber disruption. The processing system 120 may detect indicators of zonular instability by evaluating the three-dimensional rhexis edge centroid displacement from a patient-specific baseline established during an early stable phase of the surgical procedure, the capsular bag elevation derived from the three-dimensional position of the rhexis edge, the standard deviation of the distance between the rhexis edge centroid and the pupil centroid, and/or the mean change in rhexis edge orientation angle over successive image pairs. In some embodiments, the analysis module 126 may further evaluate the three-dimensional pupil axis ratio and the pupil tilt angle relative to the imaging plane, as asymmetric elongation of the pupil in three-dimensional space may indicate that the pupil boundary, rather than appearing elliptical due to eye tilt, has undergone true anatomical deformation attributable to zonular fiber disruption. When the processing system 120 determines that these features satisfy one or more prediction conditions associated with zonular instability, the processing system 120 may generate an indication of elevated zonular instability risk for presentation on the display 130, which may prompt the surgeon to consider deployment of a capsular tension ring or other adjunctive device.

In another example, in some embodiments, the processing system 120 may predict a condition of the eye 140 corresponding to intraoperative floppy iris syndrome. Intraoperative floppy iris syndrome may be characterized by iris billowing, a tendency of the iris to prolapse toward corneal incisions, and progressive pupil constriction during the surgical procedure. The processing system 120 may detect indicators of intraoperative floppy iris syndrome by evaluating the pupil tilt angle deviation, the normalized rate of change of the pupil diameter, the deviation of the physical pupil diameter from an expected baseline diameter, the pupil-to-limbus ratio, the standard deviation of the three-dimensional pupil center position over a defined temporal window, the pupil outline confidence score, the mean displacement of the rhexis edge centroid from baseline, the mean capsular bag elevation in the depth dimension, the mean change in rhexis edge orientation angle, and/or the standard deviation of the distance between the rhexis edge centroid and the pupil centroid. In some embodiments, the prediction conditions applied by the processing system 120 for intraoperative floppy iris syndrome may be further adjusted based on patient data, including prior use of medications, patient age, and ocular pigmentation grade. Based on the above, the processing system 120 may generate an indication of elevated intraoperative floppy iris syndrome risk for presentation on the display 130 during the surgical procedure.

In another example, in some embodiments, the processing system 120 may predict a condition of the eye 140 corresponding to elevated risk arising from the proximity of the medical device 142 to the iris edge during the surgical procedure. The analysis module 126 may compute the three-dimensional position of the medical device 142 by applying stereo triangulation to the segmented instrument tip location identified in each image pair captured by the first image sensor 132 and the second image sensor 134. From the three-dimensional position of the medical device 142 and the three-dimensional coordinates of the segmented iris boundary, the analysis module 126 may derive a proximity metric representing the Euclidean distance in three-dimensional space between the tip of the medical device 142 and the nearest point on the iris edge. In some embodiments, the processing system 120 may evaluate whether observed changes in the pupil centroid position, pupil shape, and/or iris boundary are spatially correlated with the position of the medical device 142, thereby distinguishing instrument-induced iris responses from responses attributable to structural or systemic conditions of the eye 140. When the proximity metric falls below a predetermined threshold and the pupil dynamics exhibit concurrent oscillatory behavior in the three-dimensional pupil center position, the processing system 120 may generate an indication of elevated instrument-proximity risk for presentation on the display 130, which may prompt the surgeon to adjust the trajectory or operational parameters of the medical device 142 to reduce the probability of instrument-induced iris or zonular stress.

Modifications, additions, or omissions may be made to the environment 100 without departing from the scope of the present disclosure. For example, in some embodiments, the first image sensor 132 and the second image sensor 134 may be integrated into a three-dimensional heads-up display surgical visualization system rather than a traditional stereo surgical microscope. As another example, the analysis module 126 may incorporate additional segmentation targets beyond the pupil, limbus, sclera, and Rhexis edge, such as the iris surface, the scleral spur, or the tips of surgical instruments, to provide additional input features for condition prediction. As another example, the processing system 120 may be distributed across multiple computing devices, including a local GPU-accelerated workstation co-located with the surgical suite and a remote server providing model inference or data logging services. As another example, the processing system 120 may be incorporated into the medical device 142.

FIG. 1B illustrates an example display 150, arranged in accordance with at least one embodiment described in the present disclosure. The display 150 may present an image 152 of an eye 160 during the surgical procedure and may be similar to the display 130. The image 152 may be captured by the first image sensor 132, the second image sensor 134, or a combination of both image sensors during the surgical procedure.

In some embodiments, the image 152 presented on the display 150 may depict the eye 160, including anatomical structures of the eye 160 such as the iris 162 and the pupil 164. In some embodiments, the display 150 may also present a previous pupil location 166, which may represent the position or boundary of the pupil 164 at a prior point in time during the surgical procedure. The previous pupil location 166 may be superimposed on the current image 152 of the eye 160, allowing a surgeon to visually compare the current state of the pupil 164 against a prior state of the pupil 164. For example, the previous pupil location 166 may be rendered as a contour or boundary outline overlaid on the current image 152 at the location where the pupil 164 was previously detected.

In some embodiments, the display 150 may present an overlay label 170 that provides an indication of one or more predicted conditions of the eye 160. The overlay label 170 may be rendered as a text-based alert, a color-coded banner, a graphical indicator, or a combination thereof. For example, the overlay label 170 may present a message such as “Floppy Iris Risk High” when the processing system 120 predicts a condition associated with intraoperative floppy iris syndrome. As another example, the overlay label 170 may present a message indicating a risk of zonular weakness, iris prolapse, or anterior chamber instability when the processing system 120 predicts one of those conditions based on the three-dimensional features or three-dimensional dynamic features of the eye 140. In some embodiments, the overlay label 170 may be color-coded to convey a severity level of the predicted condition, such as a yellow color for a warning level and a red color for an elevated risk level.

In some embodiments, the display 150 may present additional information alongside the image 152. For example, the display 150 may present a sidebar panel that includes surgeon information, procedure parameters, and real-time numeric indicators corresponding to computed features of the eye 140. The numeric indicators may include values such as a current pupil diameter, a pupil-to-limbus ratio, a composite risk score, a rate of change of pupil diameter, or a three-dimensional pupil center stability value. In some embodiments, the display 150 may also present a panel that displays operational parameters received from the medical device 142, such as intraocular pressure, vacuum level, or aspiration flow rate.

In some embodiments, the display 150 may present directional indicators on the image 152 that convey the direction and magnitude of displacement of the eye features relative to a baseline position. For example, an arrow or vector overlay may be rendered on the image 152 to indicate that the pupil 164 has shifted asymmetrically toward one side of the eye 160, which may be associated with a predicted condition such as zonular weakness on the opposite side. As another example, the display 150 may present an overlay indicating the boundary of the capsulorhexis edge relative to the pupil 164, allowing the surgeon to assess the relative displacement between the capsulorhexis edge and the pupil centroid during the surgical procedure.

In some embodiments, the display 150 may present a threshold indicator alongside the numeric indicators or the color-coded overlay. The threshold indicator may convey whether a computed feature or composite risk score has exceeded a predetermined threshold. For example, the threshold indicator may present a percentage deviation of the current pupil diameter from a baseline pupil diameter, along with a visual indication of whether that deviation has exceeded a clinically meaningful threshold. In some embodiments, the threshold indicator may be accompanied by the overlay label 170 to provide the surgeon with both a quantitative measure of the deviation and a qualitative indication of the associated predicted condition.

Modifications, additions, or omissions may be made to the display 150 without departing from the scope of the present disclosure. For example, in some embodiments, the display 150 may present three-dimensional reconstructions of the eye 140 derived from the stereo images captured by the first image sensor 132 and the second image sensor 134. As another example, the display 150 may present a temporal graph or trend plot of one or more computed features of the eye 140 over the duration of the surgical procedure, allowing the surgeon to assess the trajectory of change in those features throughout the surgery.

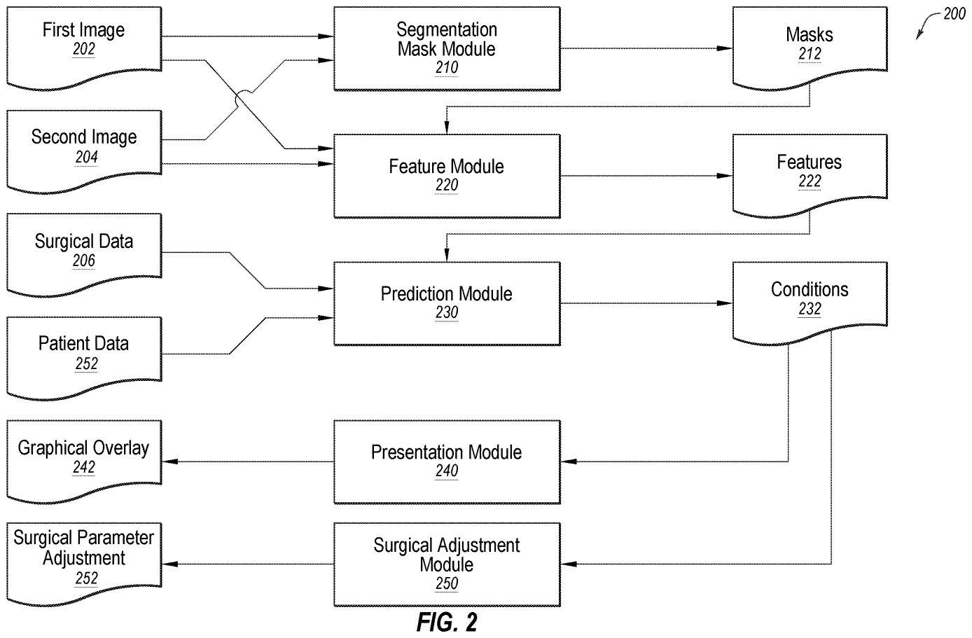

FIG. 2 illustrates an example process flow 200 for real-time, intraoperative monitoring of an eye during ophthalmic surgery. The process flow 200 may be arranged in accordance with at least one embodiment described in the present disclosure. The process flow 200 may include a segmentation mask module 210, a feature module 220, a prediction module 230, a presentation module 240, and a surgical adjustment module 250.

In some embodiments, the segmentation mask module 210, the feature module 220, the prediction module 230, the presentation module 240, and the surgical adjustment module 250 may each be a module that comprises software instructions stored in a memory, such as the memory 124, and executed by a processing system, such as the processing system 120, to perform the operations described herein with respect to each respective module.

The process flow 200 may receive a first image 202 and a second image 204 as inputs. The first image 202 and the second image 204 may correspond to the images captured by the first image sensor 132 and the second image sensor 134 of the medical system 110 described with respect to FIG. 1A. The process flow 200 may also receive surgical data 206 and patient data 208 as inputs. The surgical data 206 may include one or more parameters of the medical device 142. The patient data 208 may include information about a patient undergoing the surgical procedure.

The first image 202 and the second image 204 may each be provided as inputs to the segmentation mask module 210. The segmentation mask module 210 may apply a trained segmentation model to the first image 202 and the second image 204 to produce masks 212. The masks 212 may include segmentation masks for anatomical structures of the eye 140, such as the pupil mask, the limbus mask, the sclera mask, and/or the rhexis mask, among other masks.

The first image 202, the second image 204 and the masks 212 may each be provided as inputs to the feature module 220. The feature module 220 may apply stereo triangulation, geometric reconstruction, and temporal analysis methods to the masks 212 to extract features 222. The features 222 may include two-dimensional features, three-dimensional features, and three-dimensional dynamic features of the eye 140.

In some embodiments, the features may include pupil center calculation (X, Y, and three-dimensional position), elliptical fitting major and minor axes, pupil width and height determination, pupil diameter calculations, confidence metrics, derived geometric parameters, three-dimensional pupil center stability, three-dimensional segmentation metrics, three-dimensional pupil center and displacement measurements, three-dimensional geometric analysis of the pupil, and Rhexis parameters. Examples of how to derive each of these features from the first image 202 and the second images 204 are now provided.

In some embodiments, the pupil center calculation (X, Y, and three-dimensional position) involves a centroid (two-dimensional) calculation and three-dimensional triangulation. In the centroid (two-dimensional) calculation, for the pupil segmentation mask Mₚᵤₚᵢₗ (with pixel values 1 for pupil, 0 elsewhere), the centroid is computed as:

$${x}_{c} = \frac{{\sum }_{x , y} x * {M}_{pupil} ( x , y )}{{\sum }_{x , y} {M}_{pupil} ( x , y )}$$

$${y}_{c} = \frac{{\sum }_{x , y} y * {M}_{pupil} ( x , y )}{{\sum }_{x , y} {M}_{pupil} ( x , y )}$$

In the three-dimensional triangulation, given corresponding centroids (x_{c,L},y_{c,L}) in the left image and (x_{c,R},y_{c,R}) in the right image, the disparity may be: d=x_{c,L}−x_{c,R}. Depth Z may be calculated using:

$$Z = \frac{f * B}{d} ,$$

f, B, and d are parameters used to capture the first image 202 and the second images 204.

Then, three-dimensional coordinates of the pupil center are:

$$X = \frac{( {x}_{c , L} - {C}_{x} ) * Z}{f}$$

$$Y = \frac{( {y}_{c , L} - {C}_{y} ) * Z}{f}$$

where (cₓ,c_{y}) is the principal point.

Elliptical fitting major and minor axes may include ellipse fitting, extracted values, and axis ratio. The ellipse fitting may use a least-squares ellipse fitting algorithm (e.g., Direct Least Squares Fitting of Ellipses) on the coordinates of the pupil mask contour to obtain an ellipse:

$$\frac{{( x - {x}_{0} )}^{2}}{{a}^{2}} + \frac{{( y - {y}_{0} )}^{2}}{{b}^{2}} = 1$$

where: (x₀,y₀) us the ellipse center, 2a is the length of the major axis, and 2b is the length of the minor axis. The extracted values may include: Pupil Major Axis: Lₘₐⱼₒᵣ=2a (pixels); and Pupil Minor Axis: Lₘᵢₙₒᵣ=2b (pixels). The physical dimensions may be obtained by multiplying by s, wherein s is scale factor of mm per pixel. The axis ratio may be defined as:

$${R}_{axis} = \frac{{L}_{minor}}{{L}_{major}}$$

The pupil width and height determination may include a bounding box calculation and corresponding definitions. The bounding box determination may determine the smallest axis-aligned rectangle that contains the pupil mask. Let:

$${W}_{pupil , pixel} = {x}_{max , pupil} - {x}_{min , pupil}$$

$${H}_{pupil , pixel} = {y}_{max , pupil} - {y}_{min , pupil}$$

where Pupil Width: Wₚᵤₚᵢₗ=Wₚᵤₚᵢₗ*s; and Pupil Height: Hₚᵤₚᵢₗ=Hₚᵤₚᵢₗ*s.

The pupil diameter calculations may include a measured pupil diameter, pupil undistorted diameter, physical pupil diameter, and pupil-to-limbus ratio. For the measured pupil diameter, if the pupil is approximately circular, an effective diameter D_{measured} may be estimated as:

$${D}_{measured} = \frac{{W}_{pupil} + {H}_{pupil}}{2}$$

For the pupil undistorted diameter, to correct for optical distortion, an un-distortion algorithm may be applied using distortion parameters of the imager (e.g., radial and tangential distortion coefficients k₁, k₂, p₁, p₂, etc.) Let D_{undistorted} be the diameter computed on the undistorted segmentation mask. The exact un-distortion formula may depend on the imagers, for example:

x_{undistorted}=x(1+k₁r²+k₂r⁴)+2p₁xy+p₂(r²+2x²)

A similar formula applies for y_{undistorted}. The diameter may be recalculated using the undistorted mask.

For the physical pupil diameter, the undistorted diameter may be converted from pixels to mm:

D_{physical}=D_{undistorted}*s

The pupil-to-limbus ratio may be calculated as:

$${R}_{PL} = \frac{{D}_{physical}}{{W}_{limbus , real}}$$

Confidence metrics may include a pupil confidence and a pupil outline confidence. For the pupil confidence, the segmentation model typically produces a probability map P(x,y) for each pixel being part of the pupil. A global confidence score may be defined as the average probability over the pupil region:

$${C}_{pupil} = \frac{{\sum }_{x , y} P ( x , y ) * {M}_{pupil} ( x , y )}{{\sum }_{x , y} {M}_{pupil} ( x , y )}$$

For the pupil outline confidence, the outline confidence may be derived from the sharpness or gradient magnitude along the detected pupil boundary. For boundary pixels B, compute:

$${C}_{outline} = \frac{1}{\vert B \vert} {\sum }_{( x , y ) ∈ B} Gradient ( x , y )$$

The boundary pixels may be normalized appropriately to yield a value between 0 and 1.

In some embodiments, derived geometric parameters may include a pupil circumference and a pupil axis ratio. For the pupil circumference, using the fitted ellipse parameters, the circumference C may be approximated by Ramanujan's formula:

C≈π[3(a+b)−\sqrt{(3a+b)(a+3b)}]

where a and b may be the semi-major and semi-minor axes (in pixels). Convert to mm by multiplying by s. For the pupil axis ratio, the axis ratio R may be defined as:

$$R = \frac{b}{a} ,$$

where a value of 1 may indicate a perfect circle.

For the three-dimensional pupil center stability, variation in the three-dimensional position of the pupil center over time may indicate instability. For each frame t with three-dimensional pupil p_{3D(t)}, compute the standard deviation σP_{3D} over the surgery duration.

For the three-dimensional segmentation metrics, once a three-dimensional point cloud is generated, metrics such as volume or curvature may be calculated. For example, if the segmented pupil is represented as a three-dimensional object, its volume V may be approximated by:

$$V \approx {\sum }_{i = 1}^{N} Δ {x}_{i} Δ {y}_{i} Δ {D}_{i}$$

where Δxᵢ,Δyᵢ are the pixel dimensions (adjusted by calibration) and ΔDᵢ is the depth increment. Curvature at a point may be approximated using differential geometry methods on the three-dimensional surface.

The three-dimensional pupil center and displacement measurements may include a calculation of three-dimensional pupil center, frame-to-frame displacement and path length, and average velocity and acceleration.

For the three-dimensional pupil center, from the first image 202 and the second image 204 acquired during surgery, the pupil segmentation in each view yields two-dimensional centroids: (x_{c,L}, y_{c,L}) and (x_{c,R}, y_{c,R}), where the disparity d is given by: d=x_{c,L}−x_{c,R}.

Using the stereo calibration parameters (focal length f and baseline B), the depth Z of the pupil center may be calculated as:

$$Z = \frac{f * B}{d}$$

Then, using the intrinsic camera matrix (with principal point (cₓ, c_{y}), the three-dimensional coordinates of the pupil center from the left image may be:

$$X = \frac{( {x}_{c , L} - {c}_{x} ) * Z}{f} , Y = \frac{( {y}_{c , L} - {c}_{y} ) * Z}{f}$$

Thus, the three-dimensional pupil center is represented as: p_{3D}=(X,Y,Z).

For the frame-to-frame displacement and path length, let p_{3D}(t) be the three-dimensional pupil center at time t. The displacement vector between consecutive frames is:

ΔP_{3D}(t)=p_{3D}(t+1)−p_{3D}(t)

The Euclidean distance (displacement magnitude) for each time step is:

∥ΔP_{3D}(t)∥=\sqrt{[X(t+1)−X(t)]²+[Y(t+1)−Y(t)]²+[Z(t+1)−Z(t)]²}

The total path length Lₜₒₜₐₗ over the surgical period T (with frames indexed from t=1 to t=N) may be:

$${L}_{total} = {\sum }_{t = 1}^{N - 1} ‖ Δ {P}_{3 D} ( t ) ‖$$

For the average velocity and acceleration, the average speed v_{avg} is given by:

$${v}_{a v g} = \frac{{L}_{t o t a l}}{T} .$$

For acceleration, approximate the second derivative using finite differences:

$$a ( t ) \approx \frac{‖ Δ {P}_{3 D} ( t + 1 ) ‖ - ‖ Δ {P}_{3 D} ( t ) ‖}{Δ t}$$

where Δt is the time interval between frames.

The three-dimensional geometric analysis of the pupil may use the ellipse fitted to the two-dimensional pupil contour as described above, and the three-dimensional extension of the ellipse may be obtained by projecting the contour points into three-dimensional space.

For the pupil major and minor axes in three-dimensional, once the two-dimensional ellipse parameters (semi-major axis a and semi-minor axis b) are computed from the left (or combined) view, each boundary point may be reprojected into three-dimensional space. The axes lengths in physical units (mm) may be:

L_{major,3D}=2a*s,L_{minor,3D}=2b*s

where s is the pixel-to-mm scale factor.

For the pupil tilt angle, the tilt angle θ of the pupil relative to the imaging plane may be estimated by comparing the three-dimensional normal of the fitted ellipse plane with the ideal or expected anterior segment plane. If n is the computed normal vector and n₀ is the normal of the ideal plane (typically [0,0,1]), the tilt angle may be:

$$θ = arccos ( \frac{n * {n}_{0}}{‖ n ‖ ‖ {n}_{0} ‖} )$$

For the pupil circumference and axis ratio, using Ramanujan's formula for an ellipse: Cₑₗₗᵢₚₛₑ≈Cₑₗₗᵢₚₛₑ*s and the axis ratio R is:

$$R = \frac{b}{a}$$

The rhexis parameters may include Rhexis edge centroid, Rhexis edge displacement over time, Rhexis-to-pupil center distance variation, Rhexis edge orientation and rotation, and capsular bag elevation from three-dimensional Rhexis edge analysis. For the Rhexis edge centroid, the two-dimensional centroid of the Rhexis edge may be computed from its segmentation mask:

$${x}_{r} = \frac{{\sum }_{x , y} x * {M}_{rhexis} ( x , y )}{{\sum }_{x , y} {M}_{rhexis} ( x , y )} , {y}_{r} = \frac{{\sum }_{x , y} y * {M}_{rhexis} ( x , y )}{{\sum }_{x , y} {M}_{rhexis} ( x , y )}$$

This gives the position (xᵣ,yᵣ) of the Rhexis edge.

For the Rhexis edge displacement over time, for each frame t, compute the Rhexis centroid (xᵣ(t),yᵣ(t)). Define the baseline Rhexis center as the centroid at an early stable phase t₀. Then the displacement at time t is:

ΔR(t)=\sqrt{[xᵣ(t)−xᵣ(t₀)]²+[yᵣ(t)−yᵣ(t₀)]²}

The total or average displacement over time may be used as a metric of capsular bag instability.

For the Rhexis-to-pupil center distance variation, the two-dimensional pupil centroid may be (xₚ,yₚ) (calculated as in previous sections). The distance from the Rhexis edge centroid to the pupil center in frame t may be:

Dᵣₚ(t)=\sqrt{[xᵣ(t)−xₚ(t₀)]²+[yᵣ(t)−yₚ(t₀)]}

The standard deviation σDᵣₚ of Dᵣₚ(t) may be calculated over a series of frames. An increased σDᵣₚ may indicate that the Rhexis edge is shifting relative to the pupil, suggesting zonular weakness.

For the Rhexis edge orientation and rotation, an ellipse may be fitted to the Rhexis edge contour using a least-squares method. The fitted ellipse provides an orientation angle φᵣ(t) (the angle of the major axis with respect to the horizontal). Baseline Orientation: φ_{r,baseline} is from an early stable frame. Rotation Variation is provided by:

Δφᵣ(t)=φᵣ(t)−φr,baseline

A significant change in Δφᵣ overtime may indicate a rotation of the capsular bag edge.

For the capsular bag elevation from three-dimensional Rhexis edge analysis, using stereo reconstruction, the three-dimensional position of the Rhexis edge may be obtained. For each edge point Pᵣₕₑₓᵢₛ(t)=(Xᵣ,Yᵣ,Zᵣ), evaluate the vertical component Zᵣ. An upward shift (change in Zᵣ) relative to a baseline (e.g., at t₀) may indicate that the capsular bag is being pulled upward.

ΔZᵣₕₑₓᵢₛ(t)=Zᵣ(t)−Zᵣ(t₀)

A consistently increasing ΔZᵣₕₑₓᵢₛ may suggest a loss of zonular support.

In some embodiments, the features 222, the surgical data 206, and the patient data 208 may each be provided as inputs to the prediction module 230. The prediction module 230 may apply one or more predictive models to the features 222, the surgical data 206, and/or the patient data 208 to produce conditions 232. The conditions 232 may include risk scores and predicted probabilities associated with one or more intraoperative or postoperative conditions of the eye.

Examples of how to determine conditions using the features 222 are now provided. For example, a pupil risk score calculation may be a condition. In these and other embodiments, a pupil risk score may be based on a two-dimensional risk score calculation, pupil reaction frequency and amplitude, two-dimensional risk modeling with regression, composite risk score based on three-dimensional dynamics, and statistical models for outcome correlation.

For the two-dimensional risk score calculation, a composite risk score Rᵣᵢₛₖ may be computed using:

$${R}_{risk} = {w}_{1} * Δ D + {w}_{2} * \frac{dD}{dt} + {w}_{3} * ( 1 - R )$$

where ΔD is the deviation from baseline diameter, dD/dt is the rate of change, and R is the axis ratio. The weights w₁, w₂ and w₃ may be determined via training or calibration. A condition may be determined in response to Rᵣᵢₛₖ exceeding a preset threshold.

For the pupil reaction frequency, let Nₑ be the number of detected pupil reaction events and T_{surgery} be the total surgery duration (in seconds). Then, the frequency f_{reaction} is:

$${f}_{reaction} = \frac{{N}_{e}}{{T}_{surgery}}$$

For the amplitude, for each reaction event, let Pₘₐₓ and Pₘᵢₙ be the maximum and minimum pupil diameters within that event. The amplitude A may be A=Pₘₐₓ−Pₘᵢₙ.

For the two-dimensional risk modeling with regression, to correlate intraoperative pupil dynamics with postoperative complications (e.g., PCME (Pseudophakic Cystoid Macular Edema) or IOL (intraocular lens) (dislocation), a regression model may be used. For instance, a Cox proportional hazards model may be defined as:

h(t|X)=h₀(t)exp(β₁X₁+β₂X₂+ . . . +βₖXₖ)

where h(t|X) is the hazard function at time t given covariates X (which may include f_{reaction},A, etc.), h₀(t) is the baseline hazard function, and βᵢ are estimated coefficients from the data. The coefficients βᵢ may represent the risk increase per unit change in the covariate. For example, a higher frequency of pupil reactions may correlate with a greater risk of postoperative complications.

For the composite risk score based on three-dimensional dynamics, a composite risk score R_{risk,3D} may combine multiple three-dimensional-derived metrics. For instance:

$${R}_{risk , 3 D} = {w}_{1} * Δ {D}_{3 D} + {w}_{2} * \frac{{dD}_{3 D}}{dt} + {w}_{3} θ + {w}_{4} σ {P}_{3 D}$$

where ΔD_{3D} is the deviation in three-dimensional pupil diameter from a baseline (calculated as the difference between current and baseline physical pupil diameter), is the rate of

$$\frac{{dD}_{3 D}}{dt}$$

is the rate of change (derivative) of the three-dimensional pupil diameter over time, θ is the tilt angle (a measure of potential zonular instability), σP_{3D} is the standard deviation (or variability) of the three-dimensional pupil center positions over time, and w₁, w₂, w₃, w₄ are weights derived from model training and/or clinical calibration.

For the statistical models for outcome correlation, to correlate these intraoperative three-dimensional metrics with clinical outcomes (e.g., PCME or IOL decentration), a multivariate regression or survival model may be used. For example, the Cox proportional hazards model:

$$h ( t \vert X ) = {h}_{0} ( t ) exp ( {β}_{1} Δ {D}_{3 D} + {β}_{2} \frac{{dD}_{3 D}}{dt} + {β}_{3} θ + {β}_{4} σ {P}_{3 D} + ⋯ )$$

where h(t|X) is the hazard function at time t, h₀(t) is the baseline hazard, βᵢ are regression coefficients indicating the risk associated with each metric, and X is the vector of three-dimensional metrics and other covariates (e.g., patient age, comorbidities).

In some embodiments, intraoperative floppy iris syndrome (IFIS) may be predicted. To predict IFIS, the following features may be considered. For example, a θ Pupil Tilt Angle may be considered. A high tilt angle may indicate that the iris is not rigidly supported, which is one of the signs of floppy iris. A tilt significantly above a threshold may suggest compromised zonular support or inherent iris floppiness.

Another feature is D_{physical} Physical Pupil Diameter. Deviations from an expected dilated pupil diameter (e.g., 8.0 mm) may indicate abnormal constriction. A significant reduction in diameter during critical phases of surgery may warn of a tendency toward miosis.

Another feature is

$$\frac{dD}{dt}$$

Rate of Change of Pupil Diameter. The derivative of pupil diameter over time quantifies how quickly the pupil is constricting. A high negative value (rapid constriction) may be indicative of IFIS.

Another feature is Cₒᵤₜₗᵢₙₑ Pupil Outline Confidence. Lower confidence in the pupil's outline (possibly due to a highly irregular or “floppy” iris) may indicate an abnormal state. Although this value depends on model calibration, very low outline confidence may signal a poorly defined edge, common in floppy iris cases.

Another feature is Rₐₓᵢₛ Pupil Axis Ratio. A value significantly different from 1 indicates elongation. In cases where the iris is floppy, the pupil may not maintain a near-circular shape. Significant distortion (values far from 1) may be a risk factor.

Another feature is three-dimensional Pupil Center Stability. Variation in the three-dimensional position of the pupil center over time may indicate instability. For example, high variability in p_{3D} (i.e., a large σP_{3D}) is associated with a less stable iris.

Another feature is R_{PL} Pupil-to-Limbus Ratio. A ratio below an expected threshold (e.g., 0.70) may indicate inadequate dilation relative to the eye size, which may be a predisposing factor for IFIS. Other feature includes ΔRᵣₕₑₓᵢₛ: Average displacement of the Rhexis edge centroid from baseline (in mm), ΔZᵣₕₑₓᵢₛ: Average upward movement of the Rhexis edge (in mm), Δφᵣ: Average change in Rhexis edge orientation (in radians), σDᵣₚ: Standard deviation of the distance from Rhexis edge to pupil center (in mm).

Using the above features, to predict the risk of IFIS, the features may be combined into a composite risk score Rᵣᵢₛₖ. One formulation may include:

$${R}_{risk} = {w}_{1} Δ θ + {w}_{2} \frac{\vert \frac{d D}{d t} \vert}{{D}_{expected}} + {w}_{3} Δ D + {w}_{4} ( 1 - {R}_{P L} ) + {w}_{5} σ {P}_{3 D} + {w}_{6} ( 1 - {C}_{outline} ) + {w}_{7} Δ {R}_{rhexis} + {w}_{8} Δ {Z}_{rhexis} + {w}_{9} \vert {Δϕ}_{r} \vert + {w}_{10} σ {D}_{rp}$$

where Δθ=θ−θ_{baseline} (With θ in radians; for instance, using θ_{baseline}=20° or 0.3451 rad; dD/dt is the average rate of pupil diameter change (absolute value used to capture rapid constriction), normalized by the expected diameter; ΔD=D_{expected}−D_{physical}; R_{PL} is the pupil-to-limbus ratio; σp_{3D} is the standard deviation of the three-dimensional pupil center over time; Cₒᵤₜₗᵢₙₑ is the pupil outline confidence (scaled between 0 and 1); ΔRᵣₕₑₓᵢₛ is Average displacement of the Rhexis edge centroid from baseline; ΔZᵣₕₑₓᵢₛ is Average upward movement of the Rhexis edge; Δφᵣ is Average change in Rhexis edge orientation; σDᵣₚ is Standard deviation of the distance from Rhexis edge to pupil center; w₁, w₂, . . . , w₁₀ are weight parameters determined through clinical training and/or retrospective analysis.

In some embodiments, a risk score above a predetermined threshold (for example, 1.0) would indicate an elevated risk of IFIS. A high 40 may indicate significant pupil tilt. A high dD/dt may indicate that the pupil is smaller than expected. A low R_{PL} may suggest inadequate dilation relative to the eye size. A high σP_{3D} may reflect unstable pupil center positioning. A high ΔRᵣₕₑₓᵢₛ indicates that the Rhexis edge is moving considerably over time. A high σDᵣₚ may signify variability in the distance between the Rhexis edge and the pupil center. A high |Δφᵣ| may imply significant rotation or misalignment of the Rhexis edge. A high ΔZᵣₕₑₓᵢₛ may show upward movement of the capsular bag (potentially indicating zonular dehiscence). Thus, these features in combination, such as using the formulation above or some other formulation or model, may predict IFIS.

The conditions 232 may be provided as an input to the presentation module 240. The presentation module 240 may generate a graphical overlay 242 based on the conditions 232. The graphical overlay 242 may include colored contour lines, numeric readouts, alert banners, and other visual indicators rendered on the live surgical video feed for presentation to the surgeon during the surgical procedure.

The conditions 232 may also be provided as an input to the surgical adjustment module 250. The surgical adjustment module 250 may generate a surgical parameter adjustment 252 based on the conditions 232. The surgical parameter adjustment 252 may represent a modification to one or more parameters of the medical device participating in the surgery in response to the predicted conditions of the eye.

Modifications, additions, or omissions may be made to the process flow 200 without departing from the scope of the present disclosure.

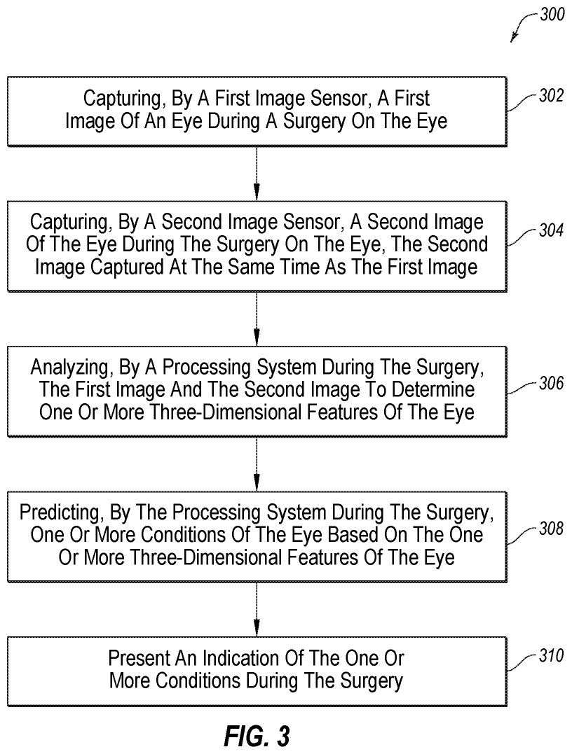

FIG. 3 illustrates a flowchart of an example method 300 for monitoring during a medical procedure. The method 300 may be arranged in accordance with at least one embodiment described in the present disclosure. One or more operations of the method 300 may be performed, in some embodiments, by a device or system, such as the medical system 110 of FIG. 1A or another device or combination of devices. In these and other embodiments, the method 300 may be performed based on the execution of instructions stored on one or more non-transitory computer-readable media. Although illustrated as discrete blocks, various blocks may be divided into additional blocks, combined into fewer blocks, or eliminated, depending on the desired implementation.

The method 300 may begin at block 302, where a first image of an eye may be captured by a first image sensor during a surgery on the eye. At block 304, a second image of the eye may be captured by a second image sensor during the surgery on the eye, with the second image captured at the same time as the first image.

At block 306, the first image and the second image may be analyzed by a processing system during the surgery to determine one or more three-dimensional features of the eye. At block 308, one or more conditions of the eye may be predicted by the processing system during the surgery based on the one or more three-dimensional features of the eye.

In some embodiments, the predicting of the one or more conditions of the eye at block 308 may be further based on one or more of: surgical data that includes a parameter of a medical device being used during the surgery and patient data that includes information about a patient undergoing the surgery. In these and other embodiments, in response to the prediction of the one or more conditions, the parameter of the medical device may be adjusted.

At block 310, an indication of the one or more conditions may be presented during the surgery. In some embodiments, the indication of the one or more conditions during the surgery may be presented concurrently with images captured by the first image sensor and the second image sensor of the eye during the surgery.

It is understood that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

For example, the method 300 may further include capturing, by the first image sensor, first images of the eye during the surgery, wherein the first images include the first image. The method 300 may also include capturing, by the second image sensor, second images of the eye during the surgery. In these and other embodiments, the second images may include the second image, and each of the second images may have a corresponding one of the first images and each corresponding pair of images may be captured at the same time. In these and other embodiments, analyzing the first image and the second image include analyzing the first images and the second images to determine one or more three-dimensional dynamic features of the eye and the one or more conditions of the eye are predicted based on the one or more three-dimensional dynamic features of the eye.

As another example, the method 300 may further include presenting, on a display during the surgery, an indication of changes in the one or more three-dimensional dynamic features of the eye over time.

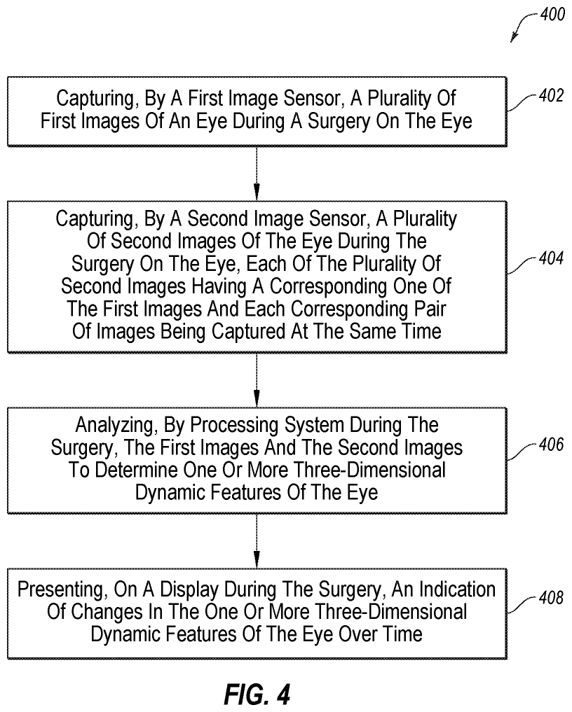

FIG. 4 illustrates a flowchart of an example method 400 for monitoring during a medical procedure. The method 400 may be arranged in accordance with at least one embodiment described in the present disclosure. One or more operations of the method 400 may be performed, in some embodiments, by a device or system, such as the medical system 110 of FIG. 1A or another device or combination of devices. In these and other embodiments, the method 400 may be performed based on the execution of instructions stored on one or more non-transitory computer-readable media. Although illustrated as discrete blocks, various blocks may be divided into additional blocks, combined into fewer blocks, or eliminated, depending on the desired implementation.

The method 400 may begin at block 402, where first images of an eye may be captured by a first image sensor during a surgery on the eye.

At block 404, second images of the eye may be captured by a second image sensor during the surgery on the eye, with each of the second images having a corresponding one of the first images and each corresponding pair of images being captured at the same time.

At block 406, the first images and the second images may be analyzed by a processing system during the surgery to determine one or more three-dimensional dynamic features of the eye. In some embodiments, the one or more three-dimensional dynamic features of the eye may relate to a pupil of the eye.

At block 408, an indication of changes in the one or more three-dimensional dynamic features of the eye may be presented on a display during the surgery over time. The display may present the indication of changes in the one or more three-dimensional dynamic features of the eye concurrently with the plurality of first images and the plurality of second images captured by the first image sensor and the second image sensor during the surgery. The indication of changes in the three-dimensional dynamic features may allow a surgeon to perceive the direction and magnitude of change in anatomical structures of the eye throughout the surgical procedure.

It is understood that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

In some embodiments, the method 400 may further include predicting, during the surgery, one or more conditions of the eye based on the one or more three-dimensional dynamic features of the eye. In some embodiments, the predicting the one or more conditions of the eye may be further based on one or more of: surgical data that includes a parameter of a medical device being used during the surgery and patient data that includes information about a patient undergoing the surgery. In these and other embodiments, in response to the prediction of the one or more conditions, the parameter of the medical device may be adjusted.

In these and other embodiments, the method 400 may further include presenting an indication of the one or more predicted conditions during the surgery. The indication of the one or more conditions may be presented concurrently with the first images and the plurality of second images captured by the first image sensor and the second image sensor of the eye during the surgery.

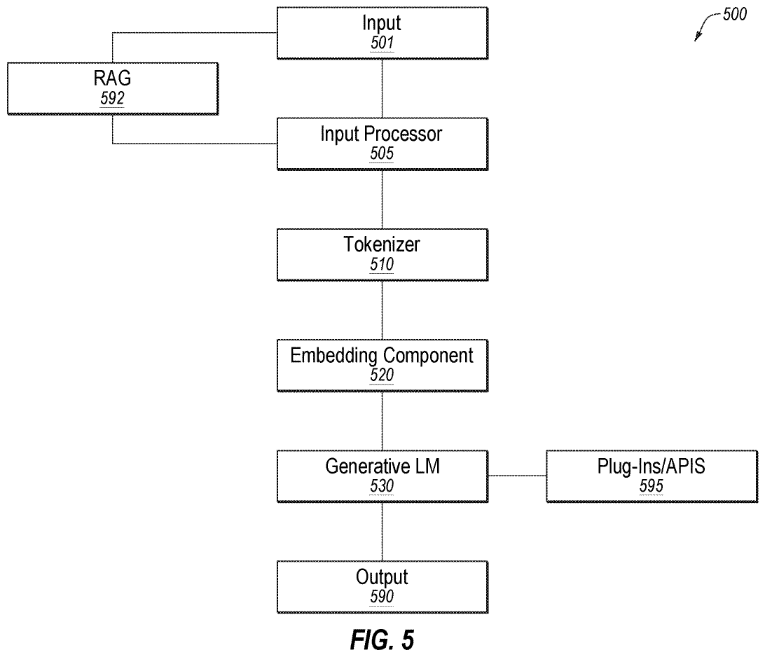

FIG. 5 is a block diagram of an example generative language model system 500 suitable for use in implementing at least some embodiments of the present disclosure. In the example illustrated in FIG. 5, the generative language model system 500 includes a retrieval augmented generation (RAG) component 592, an input processor 505, a tokenizer 510, an embedding component 520, plug-ins/APIs (application programming interfaces) 595, and a generative language model (LM) 530 (which may include an LLM (large language model), a VLM (vision language model), a MMLM (multi-modal language model), a VLA (vision language action) model, etc.). The generative language model system 500 may be an example of a system that performs the AI models in the modules described with respect to FIG. 1A.

At a high level, the input processor 505 may receive an input 501 comprising text and/or other types of input data (e.g., audio data, video data, frame data, sensor data), depending on the architecture of the generative LM 530 (e.g., LLM/LRM (Large Reasoning Model)/VLM/MMLM/etc.). Taking raw input text as an example, the input processor 505 may prepare raw input text in various ways. For example, the input processor 505 may perform various types of text filtering to remove noise (e.g., special characters, punctuation, HTML (Hypertext Markup Language) tags, stopwords, portions of an frame(s), portions of audio, etc.) from relevant textual content.

In some embodiments, a RAG component 592 (which may include one or more RAG models, and/or may be performed using the generative LM 530 itself) may be used to retrieve additional information to be used as part of the input 501 or prompt. RAG may be used to enhance the input to the LLM/LRM/VLM/MMLM/etc. with external knowledge, so that answers to specific questions or queries or requests are more relevant-such as in a case where specific knowledge is required. The RAG component 592 may fetch this additional information (e.g., grounding information, such as grounding text/frame/video/audio/USD/CAD/etc.) from one or more external sources, which may then be fed to the LLM/LRM/VLM/MMLM/etc. along with the prompt to improve accuracy of the responses or outputs of the model.

For example, in some embodiments, the input 501 may be generated using the query or input to the model (e.g., a question, a request, etc.) in addition to data retrieved using the RAG component 592. In some embodiments, the input processor 505 may analyze the input 501 and communicate with the RAG component 592 (or the RAG component 592 may be part of the input processor 505, in embodiments) in order to identify relevant text and/or other data to provide to the generative LM 530. The RAG component 592 may use various RAG techniques such as, naïve RAG, modular RAG techniques, and/or Graph RAG.

The tokenizer 510 may segment the (e.g., processed) text data into smaller units (tokens) for subsequent analysis and processing. The tokens may represent individual words, subwords, characters, portions of audio/video/frame/etc., depending on the implementation. For example, the tokenizer 510 may convert the (e.g., processed) text into a structured format according to tokenization schema being implemented in the particular embodiment.

The embedding component 520 may use any known embedding technique to transform discrete tokens into (e.g., dense, continuous vector) representations of semantic meaning. For example, the embedding component 520 may use pre-trained word embeddings (e.g., Word2Vec, GloVe, or FastText), one-hot encoding, Term Frequency-Inverse Document Frequency (TF-IDF) encoding, one or more embedding layers of a neural network, and/or otherwise. In some implementations in which the input 501 includes video data, the input processor 501 may extract frames or apply resizing to extracted frames, and the embedding component 520 may extract features such as optical flow embeddings or video embeddings and/or may encode temporal information or sequences of frames.

The generative LM 530 and/or other components of the generative LM system 500 may use different types of neural network architectures depending on the implementation. For example, transformer-based architectures such as those used in models like GPT (Generative Pre-trained Transformer) may be implemented and may include self-attention mechanisms. Some other non-limiting example architectures include transformers (e.g., encoder-decoder, decoder only, multi-modal), RNNs (Recurrent Neural Network), LSTMs (Long Short-Term Memory), fusion models, diffusion models, cross-modal embedding models that learn joint embedding spaces, graph neural networks (GNNs), hybrid architectures combining different types of architectures adversarial networks like generative adversarial networks or GANs (Generative Adversarial Network) or adversarial autoencoders (AAEs) for joint distribution learning, linear-time sequence modeling with selective state space modeling (SSM) architectures (e.g., Mamba LLM architectures), and/or others. As such, depending on the implementation and architecture, the embedding component 520 may apply an encoded representation of the input 501 to the generative LM 530, and the generative LM 530 may process the encoded representation of the input 501 to generate an output 590, which may include responsive text and/or other types of data.

As described herein, in some embodiments, the generative LM 530 may be configured to access or use- or capable of accessing or using-plug-ins/APIs 595 (which may include one or more plug-ins, application programming interfaces (APIs), databases, data stores, repositories, etc.). These and other architectures for LLMs/VLMs/MMLMs/VLAs/etc. described herein are meant simply as examples, and other suitable architectures may be implemented within the scope of the present disclosure.

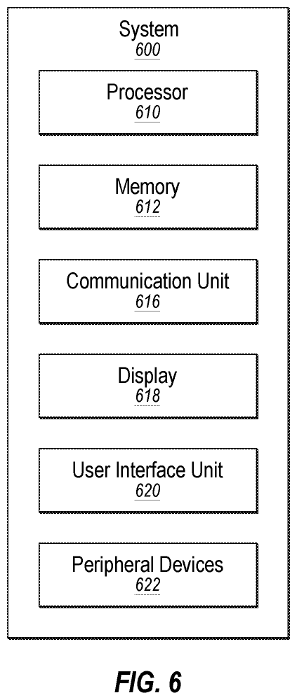

FIG. 6 illustrates an example system 600 that may be used during transcription of communications. The system 600 may be arranged in accordance with at least one embodiment described in the present disclosure. The system 600 may include a processor 610, memory 612, a communication unit 616, a display 618, a user interface unit 620, and a peripheral device 622, which all may be communicatively coupled. In some embodiments, the system 600 may be part of any of the systems or devices described in this disclosure. For example, the system 600 may be part of the processing system 120 of FIG. 1. Alternately or additionally, the system 600 may be used to perform one or more operations of the modules described with respect to FIG. 2.

Generally, the processor 610 may include any suitable special-purpose or general-purpose computer, computing entity, or processing device including various computer hardware or software modules and may be configured to execute instructions stored on any applicable computer-readable storage media. For example, the processor 610 may include a microprocessor, a microcontroller, a parallel processor such as a graphics processing unit (GPU) or tensor processing unit (TPU), a digital signal processor (DSP), an application-specific integrated circuit (ASIC), a Field-Programmable Gate Array (FPGA), quantum computer, or any other digital or analog circuitry configured to interpret and/or to execute program instructions and/or to process data.

Although illustrated as a single processor in FIG. 6, it is understood that the processor 610 may include any number of processors distributed across any number of networks or physical locations that are configured to perform individually or collectively any number of operations described herein. In some embodiments, the processor 610 may interpret and/or execute program instructions and/or process data stored in the memory 612. In some embodiments, the processor 610 may execute the program instructions stored in the memory 612.

For example, in some embodiments, the processor 610 may execute program instructions stored in the memory 612 that are related to selecting a transcription generation technique for a communication session such that the system 600 may perform or direct the performance of the operations associated therewith as directed by the instructions. In these and other embodiments, the instructions may be used to perform one or more operations of the methods described in this disclosure.

The memory 612 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 610.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media.

Computer-executable instructions may include, for example, instructions and data configured to cause the processor 610 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F.4d 1446 (Fed. Cir. 2007). Combinations of the above may also be included within the scope of computer-readable media.

The communication unit 616 may include any component, device, system, or combination thereof that is configured to transmit or receive information over a network. In some embodiments, the communication unit 616 may communicate with other devices at other locations, the same location, or even other components within the same system. For example, the communication unit 616 may include a modem, a network card (wireless or wired), an infrared communication device, a wireless communication device (such as an antenna), and/or chipset (such as a Bluetooth device, an IEEE 802.6 device (e.g., Metropolitan Area Network (MAN)), a WiFi device, a WiMax device, cellular communication facilities, etc.), and/or the like. The communication unit 616 may permit data to be exchanged with a network and/or any other devices or systems described in the present disclosure.

The display 618 may be configured as one or more displays, like an LCD (liquid crystal display), LED (light emitting diode), OLED (organic light emitting diode), Braille terminal, or other type of display. The display 618 may be configured to present video, text captions, user interfaces, and other data as directed by the processor 610. The user interface unit 620 may include any device to allow a user to interface with the system 600. For example, the user interface unit 620 may include a mouse, a track pad, a keyboard, buttons, camera, joysticks, foot pedals, and/or a touchscreen, among other devices. The user interface unit 620 may receive input from a user and provide the input to the processor 610. In some embodiments, the user interface unit 620 and the display 618 may be combined.

The peripheral devices 622 may include one or more devices. For example, the peripheral devices may include a microphone, a framer, and/or a speaker, among other peripheral devices. Modifications, additions, or omissions may be made to the system 600 without departing from the scope of the present disclosure. For example, in some embodiments, the system 600 may include any number of other components that may not be explicitly illustrated or described. Further, depending on certain implementations, the system 600 may not include one or more of the components illustrated and described.

As indicated above, the embodiments described herein may include the use of a special purpose or general-purpose computer (e.g., the processor 610 of FIG. 6) including various computer hardware or software modules, as discussed in greater detail below. Further, as indicated above, embodiments described herein may be implemented using computer-readable media (e.g., the memory 612 of FIG. 6) for carrying or having computer-executable instructions or data structures stored thereon.

Generally speaking, network environments suitable for use in implementing embodiments of the disclosure may include one or more client devices, servers, network attached storage (NAS), other backend devices, and/or other device types. The client devices, servers, and/or other device types (e.g., each device) may be implemented on one or more instances of the computing systems(s) 600 of FIG. 6—e.g., each device may include similar components, features, and/or functionality of the computing systems(s) 600. In addition, where backend devices (e.g., servers, NAS, etc.) are implemented, the backend devices may be included as part of a data center (such as, but not limited to, those described herein).

Components of a network environment may communicate with each other via a network(s), which may be wired, wireless, or both. The network may include multiple networks, or a network of networks. Compatible network environments may include one or more peer-to-peer network environments—in which case a server may not be included in a network environment- and one or more client-server network environments—in which case one or more servers may be included in a network environment. In peer-to-peer network environments, functionality described herein with respect to a server(s) may be implemented on any number of client devices.

In at least one embodiment, a network environment may include one or more cloud-based network environments, a distributed computing environment, a combination thereof, etc. A cloud-based network environment may be private (e.g., limited to a single organization), may be public (e.g., available to many organizations), and/or a combination thereof (e.g., a hybrid cloud environment).

The various features illustrated in the drawings may be, but are not necessarily, drawn to scale. The illustrations presented in the present disclosure are not meant to be actual views of any particular apparatus (e.g., device, system, etc.) or method but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” among others).

Relative terms used in the present disclosure and especially in the appended claims (e.g., bodies of the appended claims) are generally intended as falling within manufacturing tolerances and/or within scope reasonably understood by a person of skill in the art. For example, if two components are identified as being the “same” size, there may be variations consistent with manufacturing variances. Terms describing “approximately,” “similar,” “substantially,” or other terms designating similarity may convey within ten percent of the comparative value. For example, two components that are approximately the same size would be understood to be of a size within ten percent of each other.

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited in the present disclosure are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method of monitoring during a medical procedure, the method comprising:
capturing, by a first image sensor, a first image of an eye during a surgery on the eye;
capturing, by a second image sensor, a second image of the eye during the surgery on the eye, the second image captured at the same time as the first image;
analyzing, by a processing system during the surgery, the first image and the second image to determine one or more three-dimensional features of the eye;
predicting, by the processing system during the surgery, one or more conditions of the eye based on the one or more three-dimensional features of the eye; and
presenting an indication of the one or more conditions during the surgery.

2. The method of claim 1, further comprising:
capturing, by the first image sensor, a plurality of first images of the eye during the surgery, wherein the plurality of first images includes the first image; and
capturing, by the second image sensor, a plurality of second images of the eye during the surgery, wherein the plurality of second images includes the second image, each of the plurality of second images having a corresponding one of the first images and each corresponding pair of images being captured at the same time,
wherein analyzing the first image and the second image include analyzing the first images and the second images to determine one or more three-dimensional dynamic features of the eye and the one or more conditions of the eye are predicted based on the one or more three-dimensional dynamic features of the eye.

3. The method of claim 2, further comprising presenting, on a display during the surgery, an indication of changes in the one or more three-dimensional dynamic features of the eye over time.

4. The method of claim 1, wherein the indication of the one or more conditions during the surgery is presented concurrently with images captured by the first image sensor and/or the second image sensor of the eye during the surgery.

5. The method of claim 1, wherein the predicting the one or more conditions of the eye is further based on one or more of: surgical data that includes a parameter of a medical device being used during the surgery and patient data that includes information about a patient undergoing the surgery.

6. The method of claim 5, wherein in response to the prediction of the one or more conditions, the parameter of the medical device is adjusted.

7. The method of claim 1, wherein the one or more three-dimensional features of the eye relate to a pupil of the eye.

8. A medical system comprising:
a first image sensor configured to capture a first image of an eye during a surgery on the eye;
a second image sensor configured to capture a second image of the eye during the surgery on the eye, the second image captured at the same time as the first image;
a display configured to present one or more of the first image and the second image; and
one or more processors to:
analyze, during the surgery, the first image and the second image to determine one or more three-dimensional features of the eye;
predict, during the surgery, one or more conditions of the eye based on the one or more three-dimensional features of the eye; and
direct an indication of the one or more conditions to the display for presentation concurrently with images captured by the first image sensor and/or the second image sensor of the eye during the surgery.

9. The medical system of claim 8, wherein
the first image sensor is configured to capture a plurality of first images of the eye that includes the first image, and
the second image sensor is configured to capture a plurality of second images of the eye that includes the second image,
wherein analyzing the first image and the second image include analyzing the first images and the second images to determine one or more three-dimensional dynamic features of the eye and the one or more conditions of the eye are predicted based on the one or more three-dimensional dynamic features of the eye.

10. The medical system of claim 9, wherein the one or more processors directs an indication of changes in the one or more three-dimensional dynamic features of the eye over time to the display.

11. The medical system of claim 8, wherein the indication of the one or more conditions during the surgery is presented concurrently with images captured by the first image sensor and/or the second image sensor of the eye during the surgery.

12. The medical system of claim 8, further comprising a medical device used in the surgery of the eye that includes a parameter,
wherein the prediction of the one or more conditions of the eye is further based on one or more of: surgical data that includes the parameter of the medical device and patient data that includes information about a patient undergoing the surgery.

13. The medical system of claim 12, wherein in response to the prediction of the one or more conditions, the parameter of the medical device is adjusted.

14. The medical system of claim 8, wherein the one or more three-dimensional features of the eye relate to a pupil of the eye.

15. A method of monitoring during a medical procedure, the method comprising:
capturing, by a first image sensor, a plurality of first images of an eye during a surgery on the eye;
capturing, by a second image sensor, a plurality of second images of the eye during the surgery on the eye, each of the plurality of second images having a corresponding one of the first images and each corresponding pair of images being captured at the same time;
analyzing, by a processing system during the surgery, the first images and the second images to determine one or more three-dimensional dynamic features of the eye; and
presenting, on a display during the surgery, an indication of changes in the one or more three-dimensional dynamic features of the eye over time.

16. The method of claim 15, further comprising predicting, by the processing system during the surgery, one or more conditions of the eye based on the one or more three-dimensional dynamic features of the eye.

17. The method of claim 16, further comprising presenting an indication of the one or more conditions during the surgery.

18. The method of claim 16, wherein the predicting the one or more conditions of the eye is further based on one or more of: surgical data that includes a parameter of a medical device being used during the surgery and patient data that includes information about a patient undergoing the surgery.

19. The method of claim 18, wherein in response to the prediction of the one or more conditions, the parameter of the medical device is adjusted.

20. The method of claim 15, wherein the one or more three-dimensional dynamic features of the eye relate to a pupil of the eye.
